(19) United States
(12) Patent Application Publication
(10) Pub. No.: US 2025/0281439 A1
(43) Pub. Date: Sep. 11, 2025

(54) USE OF L-ASPARTIC ACID BETA-HYDROXAMATE (L-ABH) IN PREPARATION OF DRUG FOR DIABETIC RETINOPATHY (DR) OR RETINOPATHY OF PREMATURITY (ROP)

(71) Applicant: Wenzhou Medical University, Wenzhou (CN)

(72) Inventors: Shengzhou WU, Wenzhou (CN); Haiyan JIANG, Wenzhou (CN); Piansi ZHOU, Wenzhou (CN); Kaifan PEI, Wenzhou (CN); Xue JIANG, Wenzhou (CN); Jia QU, Wenzhou (CN)

(73) Assignee: Wenzhou Medical University, Wenzhou (CN)

(21) Appl. No.: 19/011,267

(22) Filed: Jan. 6, 2025

(30) Foreign Application Priority Data
Mar. 8, 2024 (CN) 202410263786.X

(51) Int. Cl. A61K 31/198; A61P 9/10

(52) U.S. Cl. CPC A61K 31/198; A61P 9/10

(57) ABSTRACT

Provided is use of L-aspartic acid β-hydroxamate (L-ABH) in preparation of a drug for diabetic retinopathy (DR) or retinopathy of prematurity (ROP). A method for protecting neural retina, comprising administrating L-aspartic acid β-hydroxamate (L-ABH) to a subject in need thereof. A method for preventing and/or treating retinopathy, comprising administrating L-ABH to a subject in need thereof.

### CROSS REFERENCE TO RELATED APPLICATION

This patent application claims the benefit and priority of Chinese Patent Application No. 202410263786.X filed with the China National Intellectual Property Administration on Mar. 8, 2024, the disclosure of which is incorporated by reference herein in its entirety as part of the present application.

### TECHNICAL FIELD

The present disclosure belongs to the technical field of medicine, and in particular relates to use of L-aspartic acid β-hydroxamate (L-ABH) in preparation of a drug for diabetic retinopathy (DR) or retinopathy of prematurity (ROP).

### BACKGROUND

Diabetic retinopathy (DR) is one of the most common complications of diabetes. Almost all patients with type I diabetes and more than 60% of patients with type II diabetes suffer from DR after a course of diabetes for more than 10 years. The DR is not only a retinal microvascular lesion, but also a retinal neurodegenerative disease. The DR is a highly blinding fundus disease with characteristic pathological changes including degenerative changes of retinal ganglion cells (RGCs) and the retinal microvascular lesions. Neuronal degenerative pathological changes include death of RGCs and neurons in inner and outer nuclear layers of retina. Retinal microvascular changes include thickening of a retinal vascular basement membrane, destruction of blood-retinal barrier (BRB), formation of microaneurysm, upregulation of intercellular adhesion molecule (ICAM-1) expression, and death of endothelial cells and outer wall cells of blood vessels. Increasing studies have shown that in early DR in both humans and experimental animals, abnormalities in the structure and function of retinal neurons and glial cells occur earlier than the retinal microvascular lesions. Early retinal neuronal damages could lead to and exacerbate relatively late the retinal microvascular lesions. Fundus optical coherence tomography (OCT)-related imaging shows that the diabetes has already caused neurodegenerative diseases in the retina before it is accompanied by fundus microvascular lesions. Compared with healthy controls, diabetic patients have suffered from thinning of the ganglion cell layer and inner plexiform layer in macula and perimacula, as well as thinning of a peripapillary nerve fiber layer.

Currently, clinical treatment of the DR is mainly aimed at relatively late-stage microvascular lesions, and there are no effective measures to protect against early-stage lesions in neural retina. Panretinal laser photocoagulation, anti-vascular endothelial growth factor (VEGF) treatment, and vitrectomy are required for mid-to late-stage vascular lesions. However, these treatments for mid-to late-stage vascular lesions could not protect the retinal neurons, and therefore could not serve as early treatment and prevention. In addition, the current non-insulin drugs for treatment of the type II diabetes include biguanides, sulfonylureas, thiazolidinediones, DPP-4 receptor inhibitors, sodium-glucose cotransporter 2 (SGLT-2) receptor inhibitors, and glucagon-like peptide-1 (GLP-1) analogs. None of these drugs are used specifically for the treatment of the DR. Moreover, drugs currently used to treat the DR are anti-VEGF antibodies and hormone drugs, which only act on the microvascular lesion stage, have only a short-term improvement effect, and show great side effects.

L-aspartic acid β-hydroxamate (L-ABH) is a reversible serine racemase (SR) inhibitor. Previous studies have found that the L-ABH could reduce random blood glucose in db/db mice, an animal model of type II diabetes, and then significantly improve oral glucose tolerance test (OGTT) and increase sensitivity of insulin tolerance test (ITT). Meanwhile, a test shows that the L-ABH could be administered orally at 20 mg/kg/d for three months without hepatotoxicity and nephrotoxicity (patent ZL 202110755406.0). However, there is no clear report on whether the L-ABH could be used for retinal neuroprotection as well as prevention and treatment of retinopathy.

### SUMMARY

In view of this, an object of the present disclosure is to provide use of L-ABH in preparation of a drug for preventing and/or treating retinopathy, where the retinopathy is selected from the group consisting of DR and ROP.

To achieve the above object, the present disclosure provides the following technical solutions.

The present disclosure provides use of L-ABH in preparation of a drug for protecting neural retina.

The present disclosure further provides use of the L-ABH in preparation of a drug for preventing and/or treating retinopathy.

In some embodiments, the retinopathy is retinal neuron degeneration disease.

In some embodiments, the retinopathy is selected from the group consisting of DR and ROP.

In some embodiments, the L-ABH improves functions of bipolar cells and amacrine cells in retina of db/db mice.

In some embodiments, the L-ABH protects a retinal nerve fiber layer (RNFL) and RGCs of the db/db mice, and prevents loss of a retinal microvascular endothelial cell (RMEC) and retinal pericytes (RPCs) of the db/db mice.

In some embodiments, the L-ABH inhibits hypoxia-induced retinal angiogenesis.

In some embodiments, the drug further includes a pharmaceutically acceptable carrier.

In some embodiments, a dosage form of the L-ABH includes one selected from the group consisting of an oral solution and a tablet.

In some embodiments, the L-ABH has a dosage of 6 mg/kg/day to 20 mg/kg/day; and the drug has a dosing frequency of 1 time/day.

Embodiments of the present disclosure have the following beneficial effects.

In the present disclosure, it is proposed for the first time that L-ABH has an effect of protecting retinal nerves and preventing and/or treating retinopathy. Experiments are conducted using db/db mice (a leptin receptor-deficient mice using to model type II diabetes). Additionally, a hypoxia-induced retinopathy model was used to model late-stage diabetic retinopathy and ROP. It is found that the L-ABH could improve functions of bipolar cells and amacrine cells in retinas of the diabetic mice. It is also found that the L-ABH could protect an RNFL and RGCs, and inhibit retinal microangiogenesis in the hypoxia-induced retinopathy model. Therefore, the L-ABH plays an effective role in preventing and treating DR or ROP.

### BRIEF DESCRIPTION OF THE DRAWINGS

FIGS. 1A-1B show results of SR expression in the retina of the wild-type mice (WT) and the db/db mice;

FIGS. 2A-2H show results of a visual electrophysiological dark adaptation test of the mice from the WT, db/db+water, and db/db+L-ABH groups;

FIG. 3A-3D show results of a visual electrophysiological light adaptation test of the mice from the WT, db/db+water, and db/db+L-ABH groups;

FIGS. 4A and 4C show OCT scan images of the mice from the WT, db/db+water, and db/db+L-ABH groups, and FIGS. 4B and 4D show analysis results of the scan images, where FIG. 4A shows a schematic diagram of circular scan, FIG. 4B shows average values obtained from FIG. 4A, FIG. 4C shows a schematic diagram of linear scan, and FIG. 4D shows processed data obtained from FIG. 4C;

FIGS. 5A-5F show results of retinal Brn3a immunofluorescence in the mice from the WT, db/db+water, and db/db+L-ABH groups;

FIGS. 6A-6C show results of retinal digest preparation—Periodic acid-Schiff (PAS) staining and haematoxylin counterstaining in the mice from the WT, db/db+water, and db/db+L-ABH groups; and

FIGS. 7A-7C show comparative results of retinal angiogenesis in the mice from the WT, OIR+Saline, and OIR+L-ABH (ip) groups.

### DETAILED DESCRIPTION OF THE EMBODIMENTS

The present disclosure provides use of L-ABH in preparation of a drug for protecting neural retina, and use of the L-ABH in preparation of a drug for preventing and/or treating retinopathy. There is no particular limitation on a specific source of the L-ABH, and any conventional commercial product in the art may be adopted.

In some embodiments of the present disclosure, the retinopathy is retinal neuron degeneration disease, the retinopathy is also selected from the group consisting of ROP or DR, and the DR includes early-stage DR and mid-stage and late-stage DR.

In the present disclosure, experiments are conducted using a db/db mouse model and a hypoxia-induced retinopathy model. The db/db mouse model is used as an early-stage model of the DR, and the hypoxia-induced retinopathy model is used as a late-stage model of the DR and could also be used as a model of the ROP. It is found that the L-ABH could improve functions of bipolar cells and amacrine cells in retina of the db/db mouse model, protect an RNFL and RGCs, and prevent loss of an RMEC and RPCs. In the hypoxia-induced retinopathy model, the L-ABH could inhibit retinal microvascular lesion and inhibit hypoxia-induced retinal angiogenesis. Moreover, L-ABH is not hepatotoxic or nephrotoxic, and has no irritating effect on gastrointestinal tract. Long-term use of the L-ABH does not cause nausea, vomiting, diarrhea, or lactic acidosis.

In the present disclosure, the drug may use L-ABH as a sole active ingredient, or may use the L-ABH in combination with other active ingredients having retinal neuroprotective effects. In the present disclosure, the drug further includes a pharmaceutically acceptable carrier. There is no special limitation on other auxiliary ingredients contained in the drug, and the commonly used auxiliary ingredients in the field of drugs may be adopted. A dosage form of the drug includes one selected from the group consisting of an oral solution and a tablet.

In the present disclosure, the L-ABH has a dosage of 6 mg/kg/day to 20 mg/kg/day; and the drug has a dosing frequency of 1 time/day. In some embodiments, in the db/db mouse model, a dose of L-ABH administered by oral gavage is (100 mg of the L-ABH is dissolved in 30 mL of water and administered via oral gavage at a dose of 0.06 mL/10 g), at a dosing frequency of 1 time/day, for continuous 18 weeks; in an OIR model, a dose of intraperitoneal injection is 6.67 mg/kg/day (50 mg of the L-ABH is dissolved in 90 mL of water and intraperitoneally injected at a dose of 0.12 mL/10 g), at a dosing frequency of 1 time/day, for 5 continuous days.

The technical solutions provided by the present disclosure will be described in detail below in conjunction with examples, but the examples should not be understood as limiting the scope of the present disclosure.

In the following examples, all methods are conventional methods, unless otherwise specified.

All materials, reagents, and the like used in the following examples are all commercially available, unless otherwise specified.

In a specific example of the present disclosure, the L-ABH is purchased from Sigma.

### EXAMPLE 1

1. Multiple 3-month-old db/db mice and 3-month-old wild-type mice (WT) were selected. The db/db mouse could be used as an early-stage model of DR.

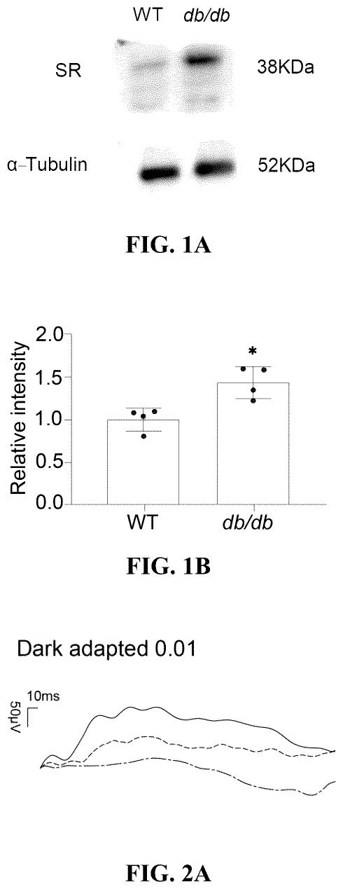

Retinas of 3-month-old db/db mice and 3-month-old wild-type mice (WT) were isolated, homogenized, and subjected to western blot (WB) analysis to detect SR expression. The results are shown in FIGS. 1A-1B. In FIGS. 1A-1B, wild-type mice (n=4), db/db mice (n=4), indicates p<0.05. As shown in FIGS. 1A-1B, SR expression in the retinas of the db/db mice is increased compared with that in the wild-type mice (WT) of the same age.

2. Verification of the protective effect of L-ABH on the retina of mice

The 3-month-old db/db mice were randomly divided into two groups and subjected to intragastric gavage experiments:
group 1: gavage of water (denoted as db/db+water, 0.06 mL/10 g, 7 mice in total);
group 2: gavage of the L-ABH (denoted as db/db+L-ABH, 20 mg/kg/day, 10 mice in total).

Each group was gavaged 1 time per day according to the dosage for 18 weeks.

The 3-month-old wild-type mice were gavaged in the same manner as the group 1 (denoted as WT+water, 0.06 mL/10 g, 6 mice in total).

(1) After the gavage, visual electrophysiological examinations were conducted on the mice in each group.

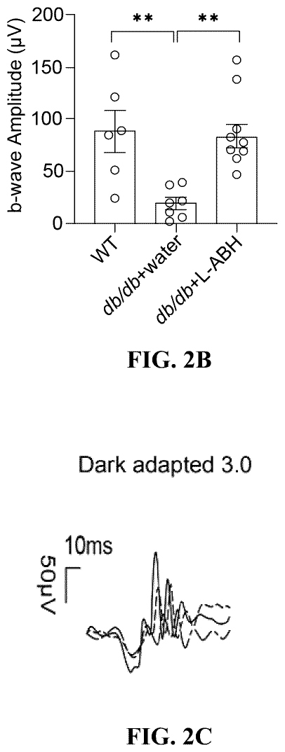

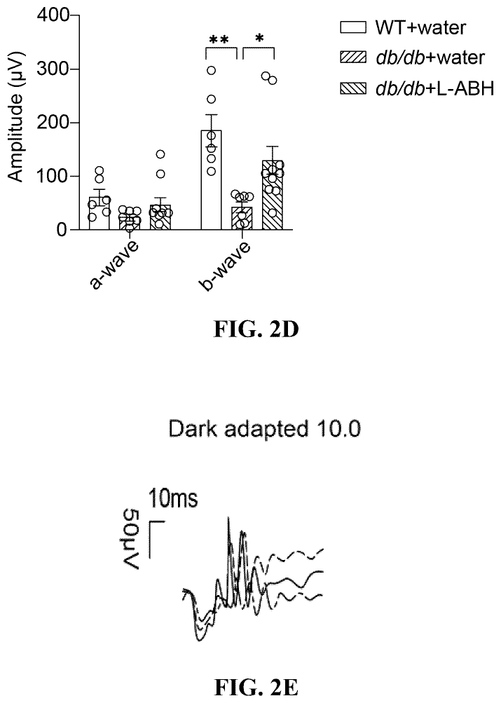

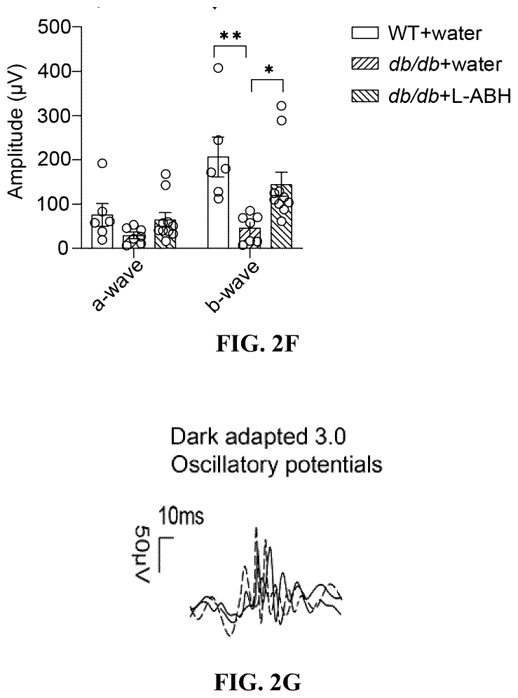

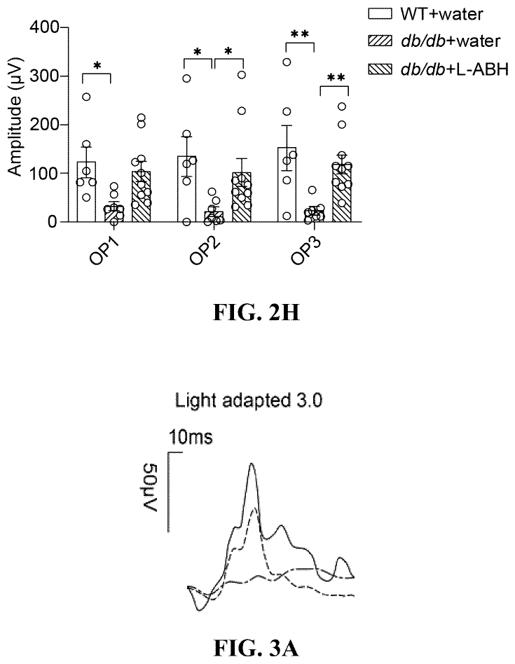

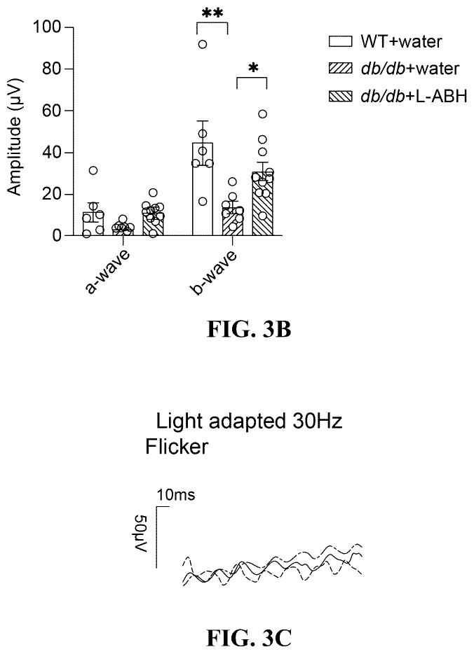

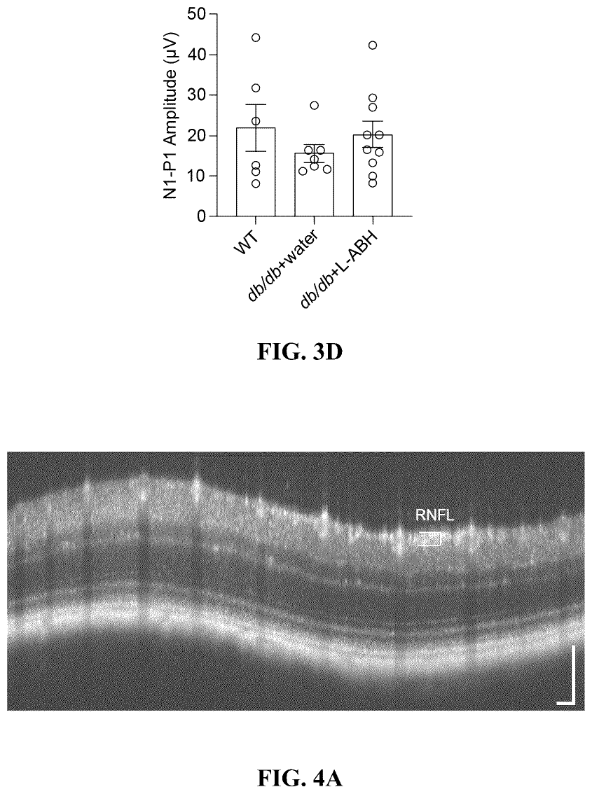

The results are shown in FIGS. 2A-2H to FIGS. 3A-3D. In FIGS. 2A-2H, (dark adapted 0.01, dark adapted 3.0, dark adapted 10.0 and dark adapted 3.0 oscillatory potentials) show 4 tests under visual electrophysiological dark adaptation. FIGS. 2A, 2C, 2E, and 2G show schematic diagrams of response curves of the 3 groups of mice, where a solid line represents the WT+water group, a dash dot line represents the db/db+water group, and a dashed line represents the db/db+L-ABH group; FIG. 2B shows a b-wave amplitude statistics graph corresponding to FIG. 2A, FIGS. 2D and 2F show a- and b-wave amplitude statistics graphs corresponding to FIGS. 2C and 2E, respectively, and FIG. 2H shows an oscillatory potential amplitude statistics graphs. In FIGS. 3A-3D, (light adapted 3.0, and light adapted 30 Hz flicker) show 2 tests after visual electrophysiological light adaptation. FIGS. 3A and 3C show schematic diagrams of response curves of the 3 groups of mice, where a solid line represents the WT+water group, a dash dot line represents the db/db+water group, and a dashed line represents the db/db+L-ABH group; FIG. 3B shows an a- and b-wave amplitude statistics graph corresponding to FIG. 3A, and FIG. 3D shows a N1-P1 wave amplitude statistics graph under 30 Hz flicker corresponding to FIG. 3C. indicates p<0.05, and “**” indicates p<0.01.

As shown in FIGS. 2A-2H and FIGS. 3A-3D, gavage of the L-ABH could significantly improve the functions of bipolar cells and amacrine cells in the retina of diabetic mice.

(2) After the gavage, an OCT experiment was conducted.

After the mice were anesthetized, tropicamide eye drops were used to dilate pupils, and after the mice entered deep anesthesia, ofloxacin was used to cover an ocular surface. The mice were placed on a mouse holder, their body position and the position knob were adjusted such that the lens was directly facing a center of mouse pupil. The position was fine-tuned from far to near such that an optic disc was located in a center of fundus image, and a focus was adjusted to make the image clear. A linear scan was conducted, where a linear scan position was moved to the vicinity of the optic disc, parameters and position were fine-tuned such that an OCT image passed through the optic disc and the optic disc was located in the middle, thus obtaining a cross-section of the retina from a temporal side to a nasal side. At the end of these operations, the images were named and saved. A circular scan was conducted, where a circular scan position was moved to vicinity of the optic disc to completely surround the optic disc, parameters and position were fine-tuned such that the OCT image was straight, thus obtaining a cross-section of the retina around the optic disc. At the end of these operations, the images were named and saved. After examining both eyes, ofloxacin was added to the ocular surface of the mice and the mice were kept warm on a temperature-constant platform until they woke up from anesthesia.

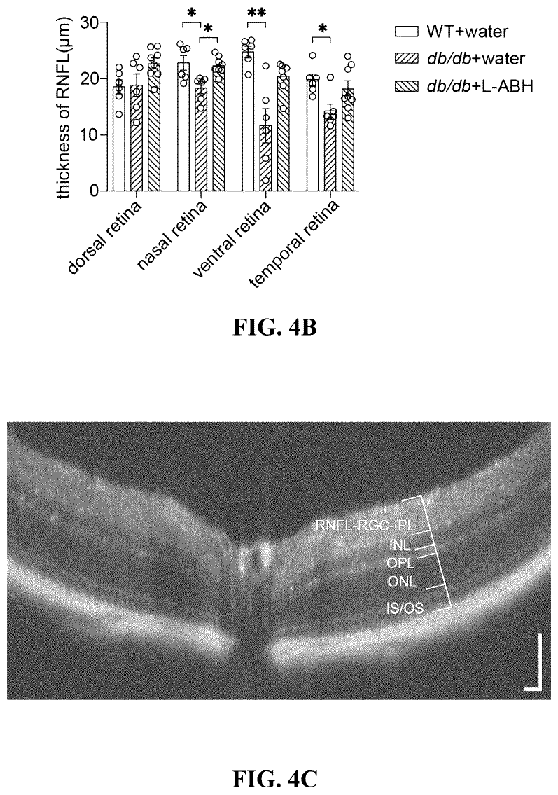

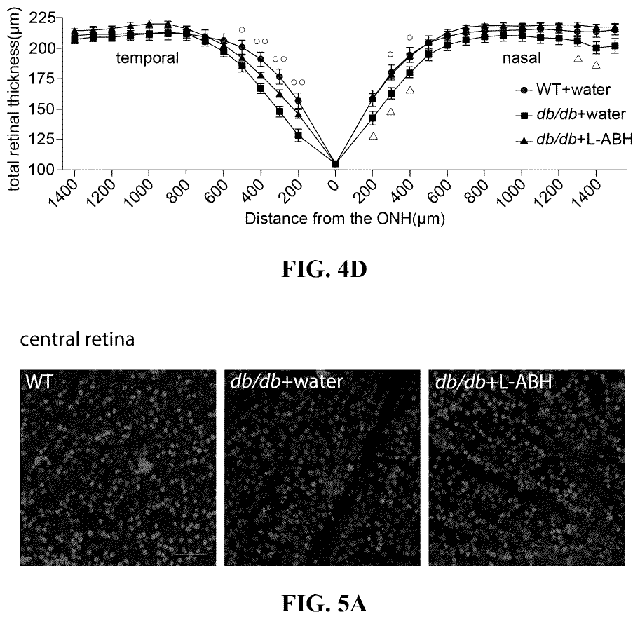

The results are shown in FIGS. 4A-4D. In FIGS. 4A-4D, FIG. 4A shows a schematic diagram of circular scan, Bar=100 μm; FIG. 4B shows an image obtained by the circular scan, which was processed and exported using insight software, and an average value of RNFL thickness in each quadrant was taken as a data point, WT+water group (n=6), db/db+water group (n=6), db/db+L-ABH group (n=8); FIG. 4C shows a schematic diagram of linear scan, Bar=100 μm; and FIG. 4D shows an image obtained by the linear scan, which was processed and exported using insight software to export data of each layer and calculate a total retinal thickness (excluding RPE). A center of the optic disc was taken as a 0 point, and a point was taken every 100 μm on both sides. In view of the fact that a structure at the optic disc had a greater impact on the data, the data at the optic disc were discarded. Combined with a width of the optic disc in the image, the data was displayed starting from 200 μm on both sides of the 0 point. ○ represents a difference between WT+water (n=8) and db/db+water (n=9) groups, and Δ represents a difference between db/db+L-ABH (n=11) and db/db+water (n=9) groups. “*” indicates p<0.05, “**” indicates p<0.01, “○” indicates p<0.05, “○○” indicates p<0.01, and “Δ” indicates p<0.05.

As shown in FIGS. 4A-4D, gavage of L-ABH could protect the degeneration of a nerve fiber layer and a medial nasal retina of db/db mice.

(3) After the gavage, retinal Brn3a immunofluorescence experiment was conducted.

After the mice were euthanized, periocular tissue of the mice was cut open, their eyeball of the mice was cut out and placed in a 5 mL centrifuge tube containing 4% PFA to allow fixation for 24 h, and the retinas were separated and cut into 4 petal shapes. The retinas were washed with PBS, permeabilized for 45 min, and blocked for 2 h. After removing a blocking solution, a Brn3a antibody was diluted (1:100) with PBS buffer containing 1% BSA, incubated overnight at 4° C., and then washed with the PBS. A fluorescent secondary antibody was diluted (1:500) with 1% BSA (prepared with PBS buffer), added to the centrifuge tube, incubated at room temperature in the dark for 1 h, and washed with PBS. The retina was transferred to a slide with a disposable pipette, the excess PBS buffer was removed with a middle pipette, the retina was spread on the slide, and the slide was sealed after adding anti-quenching agent dropwise. LSM 880 confocal laser microscope was used to take a full retinal mosaic, and then fluorescent images of the inner, middle, and outer 3 areas were intercepted on the 4 petals of the retina, that is, 12 areas of one retina were intercepted to allow fluorescence counting statistics.

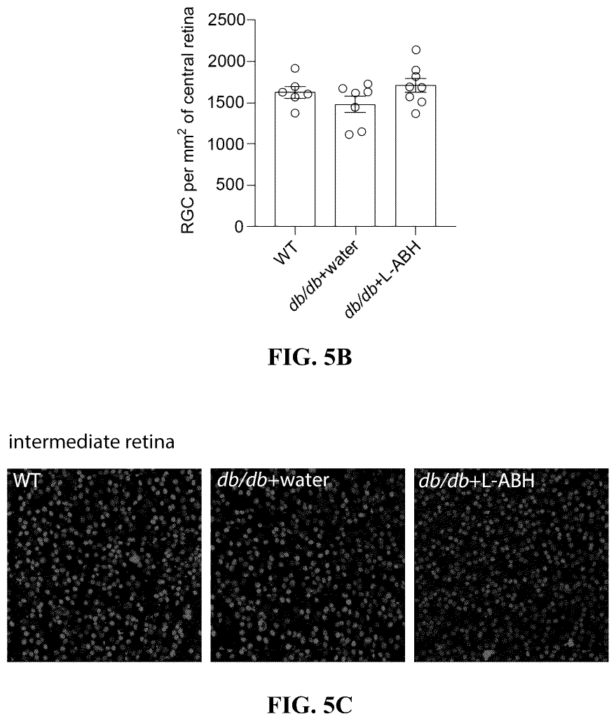

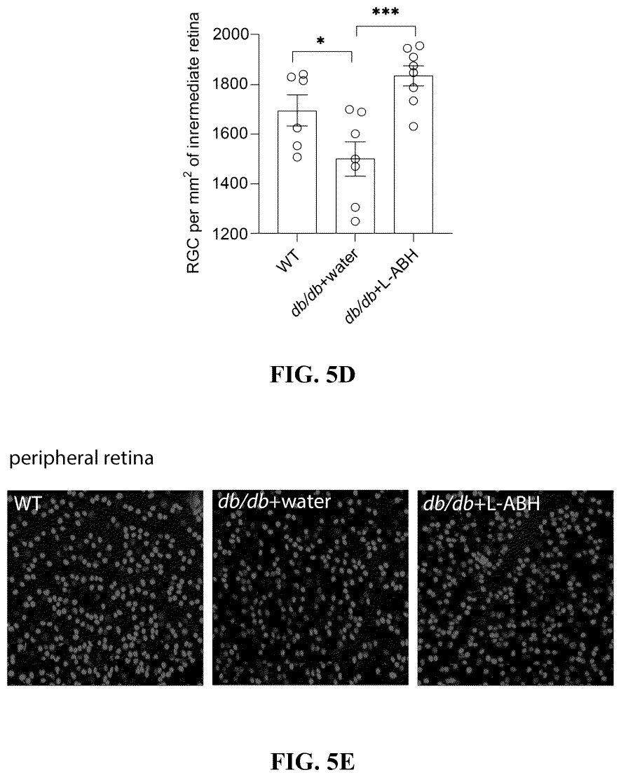

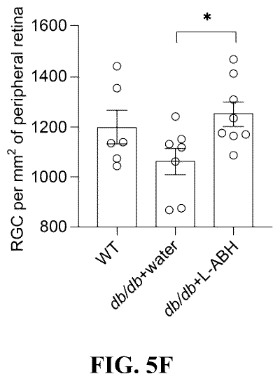

The results are shown in FIGS. 5A-5F. In FIGS. 5A-5F, from top to bottom, there are schematic diagrams and statistical graphs of the inner retina (FIGS. 5A-5B), middle retina (FIGS. 5C-5D), and outer retina (FIGS. 5E-5F) near the optic disc. FIGS. 5A, 5C, and 5E show local schematic diagrams of retinal RGCs in the WT+water group, db/db+water group, and db/db+L-ABH group of the mice (Bar=100 μm), and FIGS. 5B, 5D, and 5F show corresponding statistical graphs, where n=6 for the WT+water group, n=7 for the db/db+water group, and n=8 for the db/db+L-ABH group. “*” indicates p<0.05, and “*” indicates p<0.001.

As shown in FIGS. 5A-5F, gavage of L-ABH could significantly protect the RGCs in the middle and outer retina of db/db mice.

(4) After the gavage, the retinal digest preparation stretched preparation-PAS staining experiment was conducted.

After the mice were euthanized, eyeballs of the mice were removed and fixated with 4% PFA for 48 h, the retinas of the mice were separated and rinsed in ddH₂O on a shaker at room temperature overnight, a fixative was removed, and the retinas were cut in half. The retinas were placed in 3% trypsin (prepared in 0.2 M Tris buffer, pH=7.4) and digested at 37° C. for 1 h. Remaining retinal nerve components and internal limiting membrane were completely removed by gently pipetting, and a remaining layer of transparent retinal vascular network was moved to a slide and spread as flat as possible and then dried naturally. Retinal digest preparations were rinsed in distilled water, then oxidized in 0.15% to 1% periodic acid for 8 min, rinsed in running water for 5 min, and then rinsed 2 times in distilled water. The retinal digest preparations were stained with Schiff's solution at room temperature in the dark for 18 min, rinsed in running water for 10 min, and then rinsed in distilled water. Nuclei were counterstained with hematoxylin for 2 min and washed with running water for 10 min. The nuclei were differentiated with 1% hydrogen chloride ethanol for 3 s, then washed with tap water for 10 min, and washed with ddH₂O to make the nuclei blue again. The nuclei were washed with running water, dehydrated routinely, transparentized with xylene, sealed with neutral gum, and then 6 to 8 fields of view were randomly selected for counting and observation under a microscope, and the number of pericytes and endothelial cells was calculated.

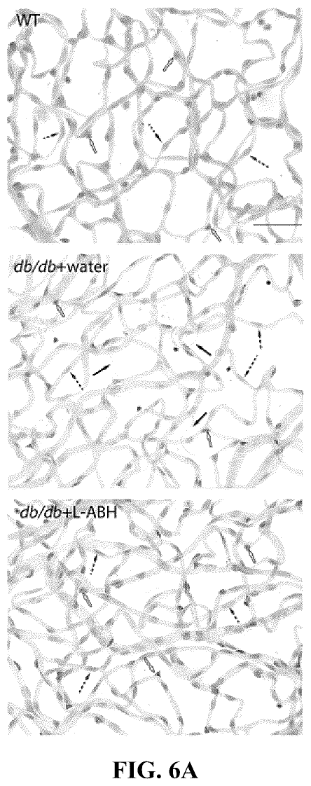

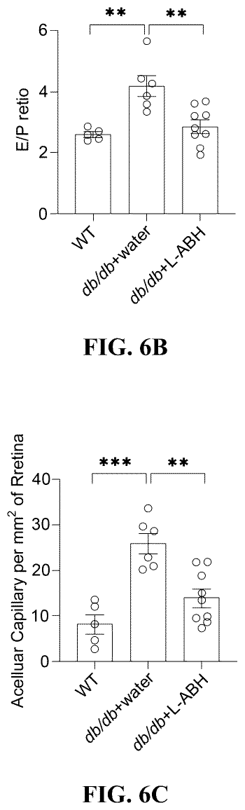

The results are shown in FIGS. 6A-6C. In FIGS. 6, FIG. 6A shows a schematic diagram of a vascular network of 3 groups of the mice, where [symbol] represents examples of endothelial cells, [symbol] represents examples of pericytes, and [symbol] represents examples of acellular capillaries, Bar=50 μm; FIG. 6B shows a statistical graph of a ratio of the endothelial cells to the pericytes; FIG. 6C shows a statistical graph of an amount of the acellular capillaries, where n=5 for the WT+water group, n=6 for the db/db+water group, and n=9 for the db/db+L-ABH group, indicates p<0.01, and “***” indicates p<0.001.

As shown in FIGS. 6A-6C, gavage of L-ABH could significantly reduce the microvascular lesions in db/db mice.

### EXAMPLE 2

In this example, the effect of L-ABH was verified in a hypoxia-induced model. The hypoxia-induced retinopathy model could be used as a model of ROP and a late-stage model of DR.

OIR experiment: neonatal mice on the 7^{th} day after birth and mother mice were randomly divided into 3 groups. Two groups of the neonatal mice and the mother mice were placed in an OIR oxygen chamber. The neonatal mice in group 1 were intraperitoneally injected with L-ABH (7 mg/kg/day) 1 time a day from the 12^{th} day after birth for 5 consecutive days, recorded as OIR+L-ABH (ip), with a total of 5 mice. While the neonatal mice in group 2 were intraperitoneally injected with an equal amount of saline every day from the 12^{th} day, recorded as OIR+Saline, with a total of 5 mice. After 5 day of hyperoxia, hypoxia-induced models were established in the group 1 and the group 2. The mice in group 3 were fed normally and intraperitoneally injected with an equal volume of saline every day, recorded as WT, with a total of 5 mice.

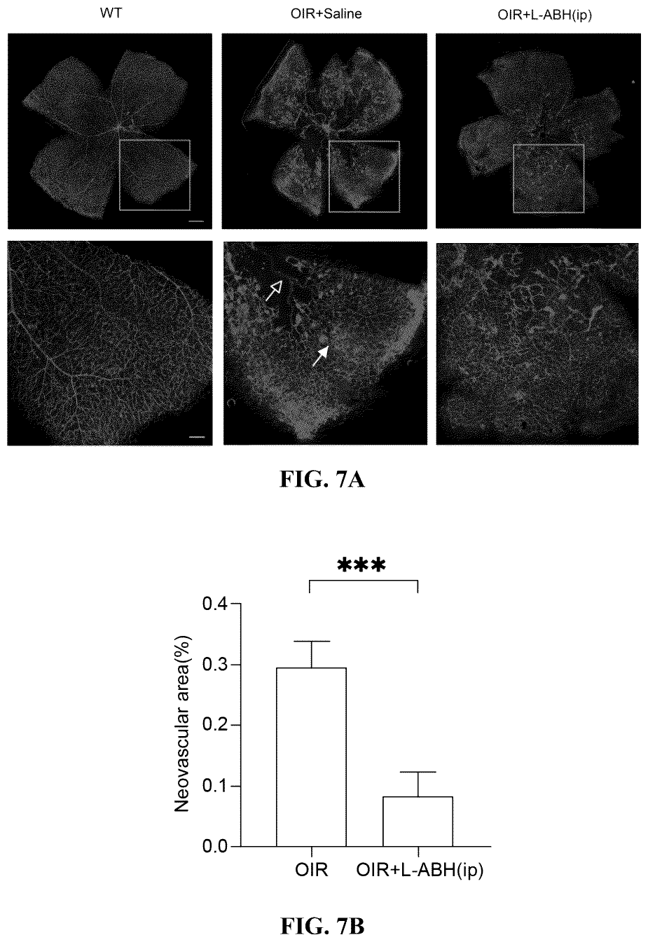

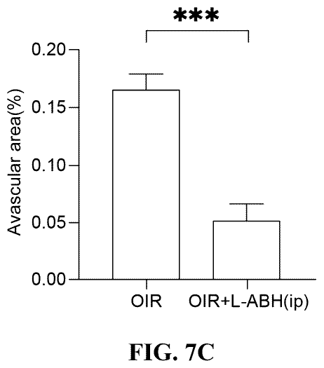

After 5 day of hyperoxia, the neonatal and the mother mice were taken out from the oxygen chamber. The neonatal and the mother mice in the 3 groups were raised normally for 5 days, then the neonatal mice were sacrificed, subjected to stretched preparation and stained with isolectin, and then analyzed for the area of retinal angiogenesis. The results are shown in FIGS. 7A-7C, where FIG. 7A in FIGS. 7A-7C shows the retinal angiogenesis, [symbol] represents an avascular area, and [symbol] represents a neovascular area. FIGS. 7B-7C are comparison of the neovascular area and the avascular area. “***” indicated p<0.001.

As shown in FIGS. 7A-7C, gavage of L-ABH could significantly reduce the hypoxia-induced retinal angiogenesis. L-ABH could alleviate the ROP.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed:

1. A method for protecting neural retina, comprising administering L-aspartic acid β-hydroxamate (L-ABH) to a subject in need thereof.

2. A method for preventing and/or treating retinopathy, comprising administering L-ABH to a subject in need thereof.

3. The method of claim 2, wherein the retinopathy is retinal neuron degeneration disease.

4. The method of claim 2, wherein the retinopathy is selected from the group consisting of diabetic retinopathy (DR) and retinopathy of prematurity (ROP).

5. The method of claim 4, wherein the L-ABH improves functions of bipolar cells and macrine cells in retina of db/db mice.

6. The method of claim 4, wherein the L-ABH protects a retinal nerve fiber layer (RNFL) and retinal ganglion cells (RGCs) of db/db mice, and prevents loss of a retinal microvascular endothelial cell (RMEC) and retinal pericytes (RPCs) of the db/db mice.

7. The method of claim 4, wherein the L-ABH inhibits hypoxia-induced retinal angiogenesis.

8. The method of claim 1, wherein the drug further comprises a pharmaceutically acceptable carrier.

9. The method of claim 1, wherein a dosage form of the L-ABH comprises one selected from the group consisting of an oral solution and a tablet.

10. The method of claim 1, wherein the L-ABH has a dosage of 6 mg/kg/day to 20 mg/kg/day; and the drug has a dosing frequency of 1 time/day.

11. The method of claim 2, wherein the drug further comprises a pharmaceutically acceptable carrier.

12. The method of claim 2, wherein a dosage form of the L-ABH comprises one selected from the group consisting of an oral solution and a tablet.

13. The method of claim 2, wherein the L-ABH has a dosage of 6 mg/kg/day to 20 mg/kg/day; and the drug has a dosing frequency of 1 time/day.
